(19) United States
(12) Patent Application Publication
(10) Pub. No.: US 2026/0007520 A1
(43) Pub. Date: Jan. 8, 2026

(54) A METHOD FOR DESIGNING AN ARTIFICIAL JOINT IMPLANT AND CORRESPONDING BROACHING AND OSTEOTOMY GUIDE APPARATUSES AND A DIGITAL IMPLANT PLATFORM THEREOF

(71) Applicant: 3 PSI LTD, Larnaka (CY)

(72) Inventor: Athanasios BATAGIANNIS, Larnaka (CY)

(21) Appl. No.: 19/125,914

(22) PCT Filed: Oct. 31, 2022
(86) PCT No.: PCT/EP2022/080355

§ 371 Date: Apr. 30, 2025

(51) Int. Cl. A61F 2/30; A61B 34/10; B22F 10/80; B33Y 50/00; B33Y 80/00; G05B 19/4099; G06T 19/20

(52) U.S. Cl. CPC A61F 2/30942; A61B 34/10; B22F 10/80; B33Y 50/00; B33Y 80/00; G05B 19/4099; G06T 19/20; A61B 2034/105; A61B 2034/108; A61F 2002/30948; A61F 2002/30952; A61F 2002/30955; A61F 2002/30985; G05B 2219/35134; G06T 2200/24; G06T 2210/41; G06T 2219/2021

(57) ABSTRACT

The present invention relates to a method for interactively designing via an implant design platform an artificial joint implant for hip arthroplasty and corresponding broaching and osteotomy guide apparatuses. The proposed implant design platform reconstructs the anatomy of the patient's pelvic and femoral bones in a 3D digital environment, and accordingly develops a patient-specific femoral implant stem as well as patient-specific surgical tools broach/rasp, osteotomy guide) used in total 10 hip arthroplasty. The patient-specific femoral implant stem developed may be optimized to ensure optimum mechanical performance for the patient, employing complex internal lattices that minimize stress shielding and advanced trabecular surfaces to promote osseointegration.

### CROSS-REFERENCE TO RELATED APPLICATIONS

This application is a national stage application filed under 37 U.S.C. 371 based on International Patent Application No. PCT/EP2022/080355, filed Oct. 31, 2022, the disclosure of which is incorporated herein by reference in its entirety.

### FIELD

The present invention relates, in general, to a computer implemented method and a digital implant design platform for interactively designing a joint replacement implant, such as a hip joint implant. Furthermore, the present invention relates to the design of a broaching apparatus and an osteotomy guide apparatus for use in preparing the area where the implant is to be inserted.

### BACKGROUND

Total Hip Arthroplasty (THA) is the most common orthopaedic surgery performed with one million surgeries performed worldwide each year—157 per 100,000 population in Europe. In THA, the head of the femur is replaced by an artificial stem and ball system, which is available in a variety of materials, such as titanium and cobalt chromium-alloys. The stem is inserted firmly into the shaft of the femoral bone and fixated either using acrylic bone cement, or by osseointegration. The wide range of stem designs available (some of them modular), allow for a strong degree of customization to account for the individual patient anatomies. The choices made, and consequently the success and longevity of the implant, however, usually depends solely on the knowledge and experience of the orthopaedic surgeon.

Therefore, there is a need for providing an implant that is custom made according to patient's femur bone anatomy thus increasing the success and longevity of the implant, while making the implant design process cheaper, faster, and more accurate. Furthermore, there is a need to develop surgical tools, such as broaching/rasp and osteotomy guiding apparatuses that correspond to the topological characteristics, e.g. geometrical dimensions and/or shape, of the patient's femur bone and/or designed joint implant.

### SUMMARY

An aim of the present invention is to provide a method and a corresponding digital implant design platform for designing an artificial joint implant that is tailored to the topological characteristics, such as geometrical dimensions and/or shape, of the patient's femur bone over a predetermined region.

The above aims are achieved according to the methods and system presented in the independent claims, while preferred embodiments are described in the dependent claims.

According to a first aspect of the present invention, a computer implemented method is provided for designing a femoral joint implant, the method comprising the steps of:
generating a three-dimensional graphic representation of a human femoral bone based on a dataset obtained from a computerised tomography, CT, scan of the human pelvis and femoral bone, the graphic representation comprising a representation of a structure of the pelvis, femur and medullary canal of the human bone where the implant is to be positioned;
processing the graphic representation of the human femoral bone to extract a set of cortical bone and medullary canal parameters comprising the shape and geometric dimensions of the medullary canal;
generating a digital model of the implant based on the extracted set of cortical bone and medullary canal parameters, the digital implant model representing an implant comprising a stem having a distal segment, a middle segment, and a proximal segment, wherein the step of generating the digital implant model comprises the step of
   adapting design characteristics of one or more segments of the stem of the digital implant model according to the set of cortical bone and medullary canal parameters such that the geometrical parameters of the one or more segments correspond to the geometrical parameters of the medullary canal over the predetermined area of the femoral bone; and
generating a set of digital implant model data representing the digital implant model with the adapted characteristics for use in the fabrication of the femoral implant.

Design of custom hip prosthesis implant, according to computer tomography (CT) images, has been previously introduced but usually relies on the use of a kinematic model of the implant joint.

The present invention is based on a modified fit-and-fill approach that takes into account the bone structure e.g. topological characteristics of the medullary canal, to design a customised joint implant, which may improve implant longevity and reduce the number of implant failures.

According to embodiments of the present invention, the generated CAD file is outputted to an additive manufacturing system e.g. 3D printer, for the manufacturing of the designed joint implant.

According to another aspect of the present invention, a method is provided for designing a broaching apparatus for performing cavity preparation of a medullary canal of a human bone where a femoral implant is to be inserted, the method comprising;
receiving design characteristics of a femoral implant designed according to method presented in the first aspect;
generating a digital model of the broaching apparatus based on a set of design parameters; and
adapting the surface of the broaching apparatus to match a surface pattern selected from a plurality of surfaces patterns, each surface pattern comprising a structure arrangement configured to compact or extract trabecular bone so as to prepare the medullary canal for implant insertion.

According to another aspect of the present invention, a method is provided for designing an osteotomy guide apparatus configured to be mounted on the femoral neck isthmus of the femoral bone, the osteotomy apparatus being adapted to match a geometry of the femoral neck so as to provide a guiding plane for the osteotomy procedure, the method comprising:
generating a three-dimensional graphic representation of a human femoral bone based on a dataset obtained from a computerised tomography, CT, scan of the human pelvis and femoral bone, the graphic representation comprising a representation of a structure of the pelvis, femur and medullary canal of the human bone where the implant designed according to claims 1 to 13 is to be positioned;
generating a digital model of the osteotomy apparatus based on a set of femoral neck cross-sections parameters extracted from the three-dimensional digital representation, the digital model comprising a fixation portion configured to surround the femoral neck; and
adapting the design characteristics of the fixation portion to match the cross-sectional geometrical dimensions and/or shape of a predetermined region along the femoral neck so as to provide a customised guiding plane during the osteotomy procedure,
wherein the fixation portion is configured to at least partially surround a portion of the predetermined region along the femoral neck, and
wherein the fixation portion comprises one or more securing elements configured to secure the osteotomy guide on the predetermined region of the femoral neck.

A digital design platform is provided for interactively designing femoral implant and corresponding broaching and osteotomy guide apparatuses according to embodiments of the present invention. The digital design platform comprises a user interface running on an electronic device and configured to interact with a user e.g. medical practitioner, during the design process of the femoral implant and corresponding broaching and osteotomy guide apparatuses. The digital design implant platform is configured to perform, by means of a processing unit and based on information and/or instructions received by the user through the user interface, the methods for designing a joint implant and corresponding surgical tools for using during the operation such as broaching and osteotomy guide apparatuses according to embodiments of the present invention.

### BRIEF DESCRIPTION OF THE DRAWINGS

The following drawings are provided as an example to explain further and describe various aspects of the invention.

FIG. 1 shows an exemplified implementation of a digital platform for designing a joint implant for use in hip replacement arthroplasty and corresponding broaching and osteotomy guide apparatuses according to embodiments of the present invention.

FIG. 2 shows an exemplified method for designing the joint implant using the digital platform according to embodiments of the present invention.

FIGS. 3a and 3b show an example of a DICOM image without annotations, and the corresponding image overlayed by the CNN's femoral cortical bone segmentation as part of the graphical representation step of the method presented in FIG. 2 according to embodiments of the present invention.

FIGS. 4a and 4b show exemplified 3D graphic representations of an intact femur bone (4a) and the same femur bone after Osteotomy along the desired osteotomy plane (x-x′) according to embodiments of the present invention.

FIG. 5 shows an exemplified digital implant mode designed according to embodiments of the present invention.

FIGS. 6a and 6b show exemplified representations of a digital joint implant model positioned within the medullary canal of the femoral bone according to embodiments of the present invention.

FIGS. 7a and 7b show exemplified representations of a designed joint implant each provided with different surface structure patterns according to embodiments of the present invention.

FIGS. 8a to 8d show cross-sectional top views of joint implants having different internal lattice structures positioned within the femoral bone medullary canal according to embodiments of the present invention.

FIGS. 9a and 9b show an exemplified representation of the Gruen zones around the femoral bone (7a) and corresponding zonal evaluation of the volume average von Mises stresses of the digital joint implant model (7b) generated according to embodiments of the present invention.

FIGS. 10a to 10c show different exemplified designs of broaching/rasp apparatuses corresponding to the geometrical dimensions and shape of the designed implant according to embodiments of the present invention.

FIG. 11 shows an exemplified implementation of an osteotomy guide apparatus positioned on the neck of the femoral bone designed according to embodiments of the present invention.

### DETAILED DESCRIPTION

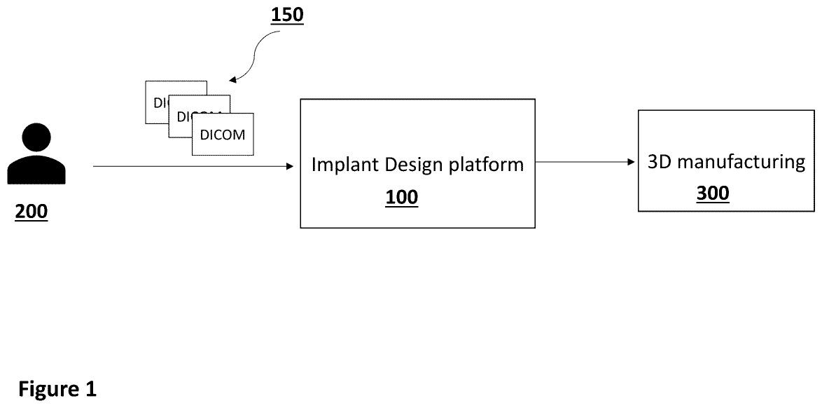

The functionality of the proposed system and method will be illustrated using the exemplified implementations shown in the FIGS. 1 to 9, which will be described in more detail below. It should be noted that any references made to specific types of data are only indicative and do not restrict the proposed functionality in any way. While the proposed system and method have been shown and described with reference to certain illustrated embodiments thereof, it will be understood by those skilled in the art that various changes in form and details may be made therein without departing the proposed functionality. Benefits and advantages of the present invention will become apparent after a careful reading of the detailed description with appropriate reference to the accompanying drawing(s).

In general the present invention provides a method and a system for interactively designing customised artificial join implants and corresponding broaching/rasp and osteotomy guide apparatuses. Accordingly a fully automated Computer-Aided Engineering customization and Finite Element Analysis optimization (FEA) platform is provided by the present invention for the design of additive-manufactured hip implants used in total hip arthroplasty. However it should be noted that design methodology may also be extended to implants used in other areas of the human body, such as the knee and shoulder joints. The proposed implant design platform reconstructs the anatomy of the patient's pelvic and femoral bones in a 3D digital environment, and accordingly develops a patient-specific femoral stem as well as patient-specific surgical tools (i.e. broach/rasp, osteotomy guide) used in total hip arthroplasty. The proposed implant design platform further optimizes the implant's design characteristics according to predetermined design parameters, and creates accompanying 3D documentation & 3D anatomical models of the hip joint, useful for preoperative planning and intra-operative guidance. The patient-specific femoral stem developed may be optimized to ensure optimum mechanical performance for the patient, employing complex internal lattices that minimize stress shielding and advanced trabecular surfaces to promote osseointegration.

According to embodiments of the present invention, the medical practitioner creates a patient profile, uploads the patient's CT scans and confirms the computed anatomical landmarks that are necessary for the restoration of the patient's hip biomechanics. Based on this information, the implant design platform reconstructs the anatomy of the patient's pelvic and femoral bones in a 3D digital environment, develops a patient-specific femoral stem as well as patient-specific surgical tools (i.e. broach/rasp, osteotomy guide) used in total hip arthroplasty, optimizes the implant's design, and creates accompanying 3D documentation & 3D anatomical models of the hip joint, useful for preoperative planning and intra-operative guidance. The patient-specific femoral stem developed may be combined with several commercial acetabular cups and femoral heads based on patient's needs and surgeon's experience. The reconstructed femoral stem, anatomical models and the surgical tools are all designed in a completely automated pipeline and produced via additive manufacturing.

Design of custom hip prosthesis implant, according to computer tomography (CT) images, has been previously introduced but usually relies on the use of a kinematic model of the implant joint. The present invention is based on a modified fit-and-fill approach that takes into account the bone internal structure to design a customised joint implant, which may improve implant longevity and reduce the number of implant failures. In general CT medical scan data is used for reconstruction of the patient's femur bone model. From the reconstructed femur bone model the anatomical shape of the medullary canal is used to design a customised joint implant having tight-fitting endoprosthesis stem over a predetermined region of the medullary canal. In essence, the stem of the joint implant is designed such that when it is inserted in the medullary canal, movement of the stem is minimised. The recommended form and dimensions of the implant are derived from the femur bone models in compliance with the needs of the patient. The fit-and-fill principle is one of the earliest design rationales in the evolution of cementless femoral stems. The fit-and-fill premise is based upon the belief that maximizing contact area of the stem with host bone would provide the greatest fixation stability and the most optimal long-term bone osseointegration with the implant. Although there has been a trend toward other design concepts, the fit-and-fill approach is still a common basis for designing cementless stems; and has been validated to be valuable in providing long-term, pain free and more suitable implant. The present invention provides a modified fit-and-fill approach for designing the joint implant to improve on its mechanical stability and longevity and help in restoring the patient's desired anatomical posture.

FIG. 1 shows an exemplified representation of a digital implant design platform 100 according to embodiments of the present invention. The implant design platform 100 is accessible by a plurality of users 200 via a user interface running on an electronic device such as a personal computer device. The users 200 of the platform may be primarily medical practitioners and/or medically trained personnel, which interact with the implant design platform 100 for the design of a customised implant for patient. However, other user types 200 may access the platform such as a patients, administrator, and the like. The digital implant design platform 100 may be a cloud-based platform accessible via a web-based application. For each patient, a medical practitioner 200 may first create a patient profile with relevant patient details, which is stored in a database of the digital implant platform. The medical practitioner may then upload datasets 150 related to Computerised Tomography (CT) scans and other image data in a Digital Imaging and Communications in Medicine (DICOM) format over a communication link. The DICOM file format is a standard protocol for the management and transmission of medical images and related patient data used in healthcare facilities such as hospital, clinics, and the likes. Based on the information provided by the user 200 and included in the DICOM dataset, the anatomy of the patient's pelvic and femoral bones is reconstructed, at a reconstruction module, in a 3D digital environment to provide a 3D reconstructed model of the pelvic and femoral bone. For the 3D reconstruction an image reconstruction machine learning convolution neural network (ML-CNN) algorithm may be used, which has been trained on image data from CT scans and configured to extract design parameters of the pelvic and femoral bones of the patient, including parameters of the medullary canal of the femoral bone where the implant is to be inserted. Accordingly, a digital joint implant design model is created having stem with design characteristics that are interactively optimised and adapted in the digital platform based on the extracted design parameters to match corresponding design characteristics of the medullary canal of the femoral bone. The design parameters may comprise 3D geometrical parameters extracted from the reconstructed 3D model of the pelvic and femoral bones by the ML-CNN and/or provided by the medical practitioner. Once the optimisation of the implant model design characteristics is finalised, a dataset associated with the optimised joint implant model is created e.g. a CAD file, which may be communicated to an additive manufacturing system 300 e.g. 3D printer, for the manufacturing of the patient specific femoral implant having a customised stem. The digital platform 200 is provided with a user interface running on an electronic device that allows the users to interact with the digital platform during the implant design process.

Accordingly, based on the reconstructed 3D model of the pelvic and femoral bones and/or the design of the optimised joint implant model, patient-specific surgical tools such customised broach/rasp apparatuses, and osteotomy guide apparatuses used in hip arthroplasty may be designed in the digital design implant platform 100. Furthermore, accompanying 3D documentation & 3D anatomical models of the hip joint, may be generated to aid the surgeon and medical staff during preoperative planning and intra-operative guidance. The patient-specific femoral stem developed may be combined with several commercial acetabular cups and femoral heads based on patient's needs and surgeon's experience. The reconstructed femoral stem, anatomical models and the surgical tools are all designed in an interactive design environment provided by the implant design platform 100 and produced via additive manufacturing.

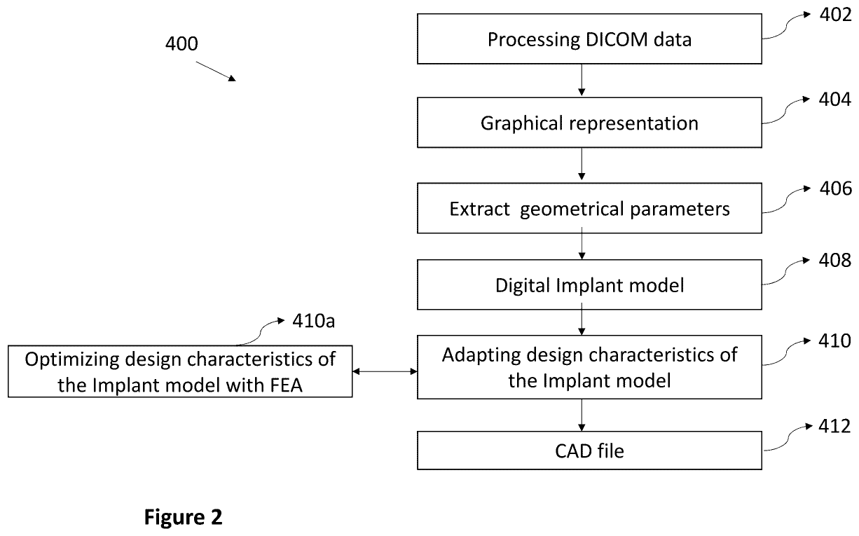

FIG. 2 shows an exemplified computer implemented method 400 for designing an artificial joint implant for hip arthroplasty according to embodiments of the present invention via the digital design implant platform 100. As previously described the DICOM datasets associated with the computer tomography (CT) scans of the patients are communicated via the user graphic interface of the implant design platform 200, where they are processed at step 402. The step of processing comprising among other operations sorting and filtering which involves anonymization and indexing, to ensure processing of the data according to established medical data standards. For security reasons the DICOM upload is handled by a secure data transfer protocol, e.g. https or similar. For privacy reasons the patient's name is removed from the dataset, a process commonly known as DICOM anonymization. The processing step 402 may comprise the step of anonymization of the DICOM files to remove sensitive patient information and only maintain information that are necessary for the design of the implant such as the CT scans along with patient information related to the height, weight, age and sex of the patient. The patient information (height, weight, age and sex), are presented to the surgeon or if absent prompted to fill-in at the user interface of the digital implant platform e.g. in the form of a menu.

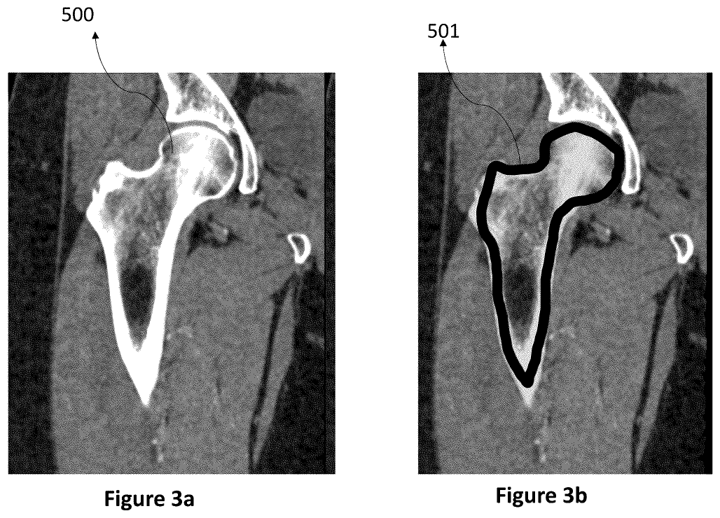

The processed DICOM datasets 150 are used in the 3D reconstruction step 404, whereby a 3D graphic representation of the human femoral bone is generated based on the computerised tomography, CT, scans, also referred to as images, of the human pelvis and femoral bone. The 3D graphic representation comprising a representation of the structure of the pelvis, femur and medullary canal of the patient's bone where the implant is to be positioned. The 3D reconstruction step 404 involves the steps of image segmentation and 3D reconstruction tasks. These tasks are executed in the background, without the user interaction. The segmentation task processes the raw DICOM series to exclude artifacts that correspond to low Housfield units (e.g. below a certain threshold, such as air and fat, and uses a 3D U-Net Convolutional Neural Network (CNN) architecture to produce segmentation masks for the femoral bone's cortical parts, as well as for the pelvis bone. The 3D U-Net CNN used for generating the image segmentation masks may be similar to the one described in “Çiçek, Ö., Abdulkadir, A., Lienkamp, S. S., Brox, T., Ronneberger, O.: 3D U-Net: learning dense volumetric segmentation from sparse annotation. In: Ourselin, S., Joskowicz, L., Sabuncu, M. R., Unal, G., Wells, W. (eds.) MICCAI 2016. LNCS, vol. 9901, pp. 424-432. Springer, Cham (2016)”. The 3D reconstruction step 404 utilizes the segmentation masks from the CNN model and the DICOM series metadata to produce triangulated meshes of the pelvis and femur's surfaces. This process is based on the marching cube algorithm. Moreover, as an optional task, a 2D projection of the DICOM series in the anterior-posterior plane is generated. This projection is computed to aid the surgeon determine the leg-length correction, if it is deemed to be required during the design process of the implant. FIGS. 3a and 3b show an exemplified DICOM image of the pelvis and femoral bone 500 without annotations, and the same image overlayed by the CNN's femoral cortical bone segmentation mask 501.

During the 3D reconstruction of the femur bone, the step of leg-length determination may be performed to correct any height discrepancy between left and right femurs. The medical practitioner e.g. surgeon, is asked via the user interface of the digital implant platform to confirm leg-length correction by showing projections of the femur in the superior-inferior and anterior-posterior views, in a similar manner to 2D X-rays. This is derived by checking the difference in pixels between the left and right lesser trochanter from a common reference point on the pelvis as defined by the medical practitioner. The difference in pixels between the images along the regions of interest is translated to metric length values e.g. millimetres, and stored. This is achieved by multiplying the difference in pixels by the DICOM spacing value provided in the DICOM dataset.

The next step in the design process involves the determination of the femur's bone key anatomical parameters and the definition of the osteotomy plane at step 406. The anatomical parameters relate to cortical bone and medullary canal parameters comprising the shape and geometric dimensions of the medullary canal. For that purpose algorithms based on Boolean operations (intersections, differences, unions and cuts) combined with Al-empowered feature detection are used to calculate the femur parameters required (anteversion, neck-shaft, trochanters, neck and femur axes and isthmuses, offset). The surgeon can confirm or modify the extracted 3D geometrical parameters through the digital implant platform 100 user interface. The 3D geometrical parameters are as follows:
Anteversion angle
Femoral and neck axes
Neck shaft angle
Femoral head centre location
Lesser and great trochanter references

As a result the system also derives (without user intervention) the following parameters
Canal flare index
Femoral and neck cross sections and radii

In general, femoral anteversion angle is defined as the angle between the femoral neck axis and the line connecting two aspects of the posterior condyles (transcondylar axis of the knee) in the transverse plane. Anatomically, it represents an internal twisting of the femur with respect to the femoral anatomical axis. This orientation of implant is often specified in terms of two angles relative to the patient anatomy, abduction, and anteversion. These angles specify rotations which transform the implant from a neutral position to the desired position. The parameter of femoral anteversion angle is crucial to design a patient-specific implant especially for abnormal patients.

According to embodiments of the present invention, the value for the anteversion angle is determined based on information extracted from the DICOM datasets and/or doctor input during the design process. The value for the anteversion angle is calculated and confirmed by the doctor.

In general, Femoral and neck cross sections and radii are defined as cross sections, centroids and mean radii at selected locations for femoral shaft and neck shaft are needed in order to define the cross sections of implant geometry so as to have good fit and fill, as described in “G. Saravana Kumar & M. Gupta, “Patient specific parametric geometric modelling of cementless hip prosthesis”, Innovative Developments in Virtual and Physical Prototyping: Proceedings of the 5th International Conference on Advanced Research in Virtual and Rapid Prototyping, Leiria, Portugal, 28 Sep.-1 Oct. 2011”.

Femoral and neck axes are obtained from various centroids and cross sections, create a new reference plane and coordinate system and aid in extraction of more features from the femur crucial for the implant design.

Neck shaft angle is obtained by slicing the femoral neck of the 3D representation of the femur bone, during step 406, in order to find the femoral neck isthmus in which the radius of the sliced spline has the smallest value. The orientation of slicing planes is 45 degrees with the X-Z plane. All the centres of the sliced splines are interpolated to obtain the femoral neck axis. The neck shaft angle is defined as the angle between the femoral and neck axis (frontal plane deformities: Coxa valga>135 degrees, coxa norma=120-135 degrees and coxa vara<120 degrees) and guarantees stable and correct adjustment between the implant, ball, acetabular and pelvis. The femoral neck shaft angle appears as the angle between the projections of the neck axis (Vn) and the anatomical femoral axis (Va). The angle can be computed using the following basic vector computation (Eq. (1)).

$$Neck shaft angle θ = {cos}^{- 1} \text{?} \qquad ( 1 )$$

$$\text{?} \text{indicates text missing or illegible when filed}$$

According to embodiments of the present invention, the correct value for the neck shaft angle is calculated and confirmed by the doctor during the design process of the implant.

Femoral head centre location and head sphere radius parameters are important as a reference location in creating the coordinate system for the femur and design or fit the correct acetabular part.

Lesser trochanter reference is defined as the plane where the lesser trochanter is placed is very critical for the fitting of the implant due to wide variations in the anatomy of the femur in the upper and lower area.

Canal flare index (CFI): The overall shape of the stem is determined with the CFI parameter

since the stem seats in the canal of the femur. This parameter is used to detect the possibility of interference during insertion of the implant and size the implant accordingly to avoid it.

In general, the 3D geometrical parameters extracted at step 406, contribute to the custom implant design and have a direct influence in the accuracy of the resulting patient-specific implant geometry

Once the femur parameters are determined the osteotomy plane is defined (through a slider or by entering the desired coordinates) and the femoral cut is finalized. This plane defines the division between the proximal part of the implant (which includes the taper and the rest of the extramedullary part) and the mid segment which is wholly implanted in the femoral canal. In general, the neck osteotomy level varies depending on the bone size of the patient, the neck angle, and preoperative templating. The osteotomy should be approximately 45 degrees to the femoral canal axis, and approximately 2 cm above the lesser trochanter.

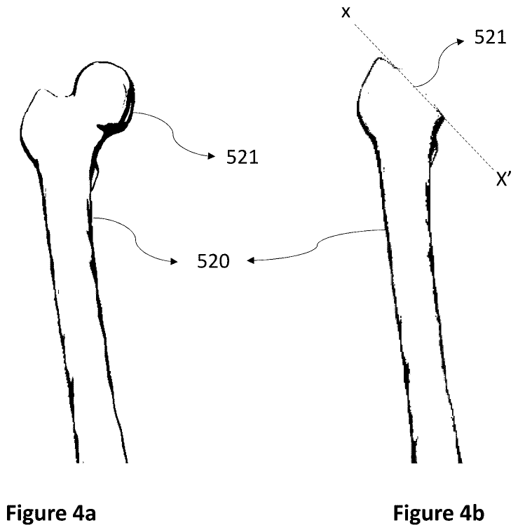

FIGS. 4a and 4b show exemplified 3D representations of the femur bone generated from the 3D graphic representation step 404. FIG. 4a show an intact femur bone 520 and the femur neck 521. In FIG. 4b, the same femur bone 520 is represented having a neck osteotomy performed along the desired osteotomy plane x-x′.

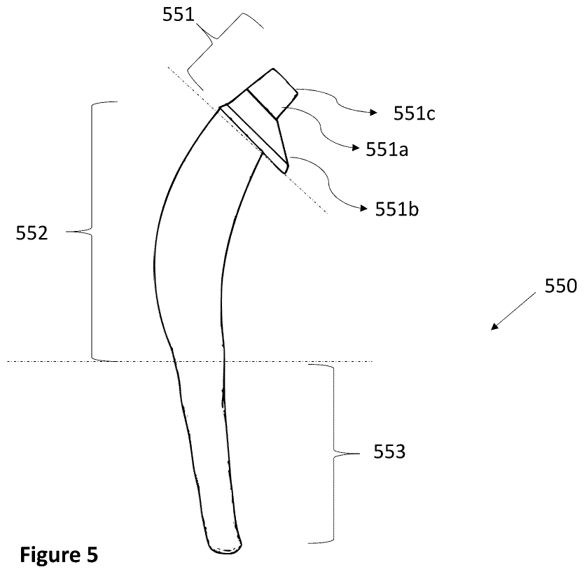

The extracted 3D parameters and the 3D representation of the femur bone are used in step 408 to generate a digital representation of an implant model according to embodiments of the present invention. The digital implant model comprises a stem 550 having a distal segment 553, a middle segment 552, and a proximal segment, as shown in FIG. 5. These segments have a specific purpose considering form and function. The distal segment 553 allows positioning of endoprosthesis body in the medullary channel and provides primary fixation and load transfer. The mid segment 552 passes through the porous part of the bone and follows the anatomical parameters of the femur medullary canal, which is crucial for osseointegration. The proximal segment 551 contains a femoral neck 551a which facilitates the placement of various sizes of femoral heads and acetabular cups, and a collar 551b, if optionally selected.

At step 410, geometrical factors of the femur bone extracted during step 406, are used in the design of the digital implant model, so as to achieve a customised fit. More in particular, the design characteristics of one or more segments of the stem 550 of the digital implant model is adapted during step 410 according to the set of cortical bone and medullary canal parameters such that the geometrical parameters of the one or more segments correspond to the geometrical parameters of the medullary canal over a predetermined area of the femoral bone. As such the desired parameters of the digital implant model are calculated and adapted at the digital implant platform 100 based on the 3D parameters extracted at step 406.

The distal segment 553 of the implant stem 550 depends on the shape and dimensions of the medullary channel 522 of the femur, also referred to as femoral bone or thigh bone. For design of the distal segment 553 the following parameters may be taken into consideration at step 410:
The position of the medullary channel isthmus
Lesser trochanter position
Layout and dimensions of the medullary canal 522, which are either preserved as measured/calculated or fitted with an ellipsoid shape.

The distal segment of the implant stem in general provides the implants proper positioning, enables the transfer of the load from the pelvic region to the foot and ensures proper positioning of the leg to improve patient comfort. The geometrical elements that define the shape and dimensions of the mid segment, and based on which the design characteristics of the mid-segment 552 are adapted, may include:
Position of the lesser trochanter
Position of the osteotomy
The dimensions of the medullary canal, also referred to as channel, at a predetermined distance, e.g. 20 mm, above and below the lesser trochanter
Canal flare index (CFI), an expansion coefficient of the medullary channel defined as the ratio of the diameter of the femoral canal at the isthmus in the anteroposterior (A-P) view to the diameter of the medullary canal 20 mm above the lesser trochanter
Neck shaft angle
Femoral anteversion angle

The proximal segment 551 consists of four parts: a body, collar 551b, neck 551a and cone upon which the femoral head is placed. The dimensions and shape of the body and collar 551b are determined based on structural conditions such as position of the femoral head, distance of the femoral head from the axis and the angle of the femoral neck. Dimensions are sized to fit the selected head and acetabular cup.

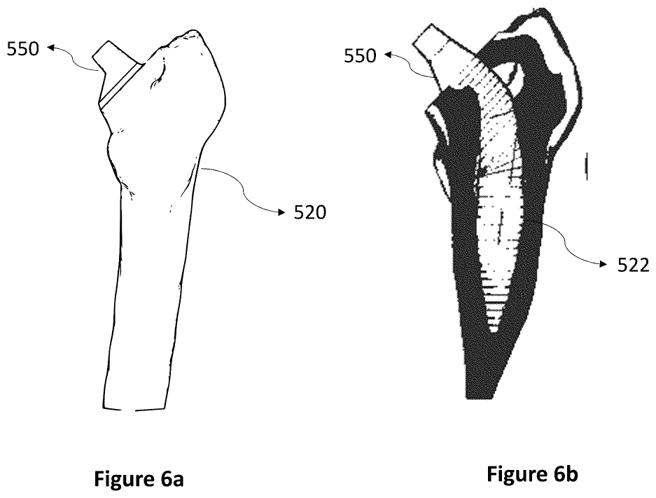

FIGS. 6a and 6b show exemplified representation of a digital implant model positioned inside the medullary canal 522 of the femur representation 520. As shown in FIG. 6b, the middle segment 552 and distal segment 553 are located within the medullary canal 522, with portion of the middle and distal segment being in contact with corresponding surfaces of the medullary canal 522. The proximal segment 551 of the implant model is extended outside of the femur 520 along the neck shaft angle extracted at step 406.

In general during step 410, the medical practitioner e.g. surgeon, may provide the following parameters:
the stem length, which may be selected to be optimised during step 410 or not,
the lateral offset, if it is desired to alter the femur's value extracted during step 406,
whether a collar 551b is required or not, which it can be optimized,
The type of desired surface structure for the mid segment 552. For example, the surface structure may be based on a trabecular pattern based on cancellous bone structure to promote osseointegration, trabecular pattern based on geometrical structures and smooth.

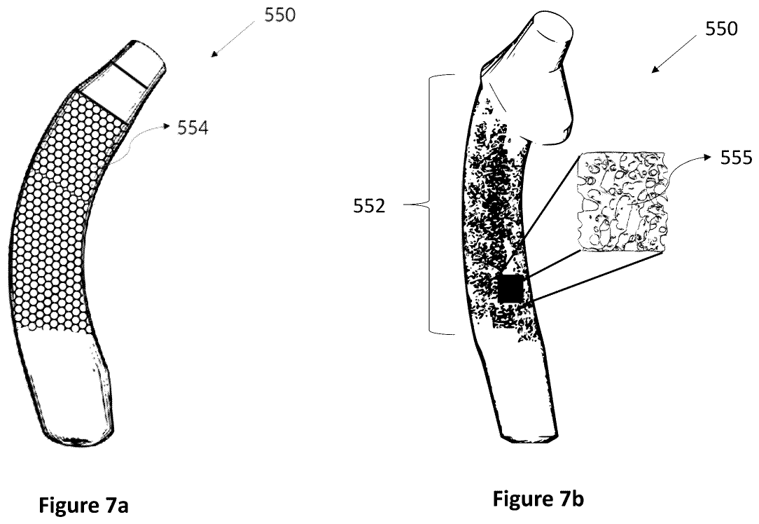

FIGS. 7a and 7b show exemplified representation of an implant having different surface design structures on the mid segment 552. The surface structures essentially represent a porous inter-connected network of holes into which cancellous cone is to grow, thus promoting implant stability through osseointegration. The holes extend to a depth of 2 mm inside the mid segment of the implant and can be geometric (e.g. circular), with a fixed size of ˜0.25 mm, or anisotropic (e.g. based on the structure of cancellous bone itself), with variable sizes and shapes. FIG. 7a shows an implant representation with a trabecular pattern 554 based on geometrical shapes. FIG. 7b shows an implant representation with a trabecular pattern 555 along the mid segment 552 based on cancellous bone structure. The cancellous trabecular structure pattern 555 may comprise cavities having a shape representative of the cancellous collagen cell structure with the intent to promote and enhance osseointegration. The density and coverage of the middle segment 552 may be selected by the practitioner and/or optimised during step 410. In general, porous or trabecular surfaces are widely used in clinical applications, including porous orthopaedic and dental implants. Interconnected pores permit tissue and bone ingrowth, preventing loosening, and retaining dynamic strength of implants. When a porous implant is implanted into the marrow cavity, fibrin and fibrous tissue are observed in the initial stage, followed by woven bone formation. Woven bone is remodelled into lamella bone and bone marrow-like tissue replaces the fibrous tissue. This is desired as it encourages osseointegration and thus optimal biological fixation. Implants are already available on the market which feature trabecular surfaces, albeit patterned with simple geometrical lattices.

During step 410, the medical practitioner may select the implant type out of the following options:
Fully custom: The distal implant segment is fully personalized, with the implant's surface for every cross section matching exactly the cross-section of the femoral canal. This means that the implant cannot be rotated in a position other than the one intended for implantation, thus making it misplacement-proof.
Semi-custom: The distal implant segment is only partially personalized in a narrow zone between the mid and distal implant segments, with the implant's surface for every cross section matching exactly the cross-section of the femoral canal only in this zone. The rest of the distal segment is approximated by ellipsoids (with equal and/or unequal arcs). This implant type it also misplacement-proof for the same reason.
Ellipsoid-type: This type follows the fit and fill approach using only ellipsoids. As such, it closely resembles the cross sections of a commercial standardised implant, following however the specific structure of the patients femoral canal.

In general, according to embodiments of the present invention, the mid implant segment may also be based on ellipsoids along the femoral and neck axis which are joined with lofts. 3D splines control the shape of the ellipsoids such that it can be morphed into any desired 3D shape. Furthermore, the cross sections can be solid ellipsoids or lattice structures, resulting in solid or hollowed mid segments respectively. The implant design parameters may be optimised using Finite Element Analysis.

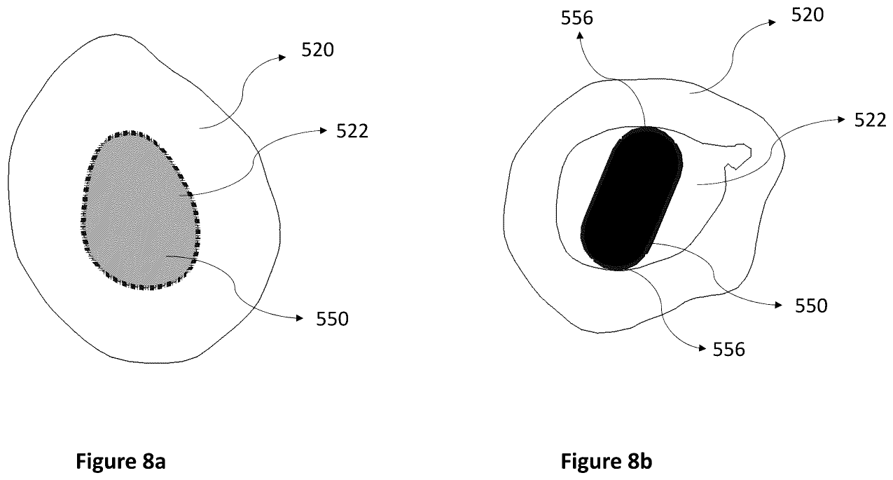

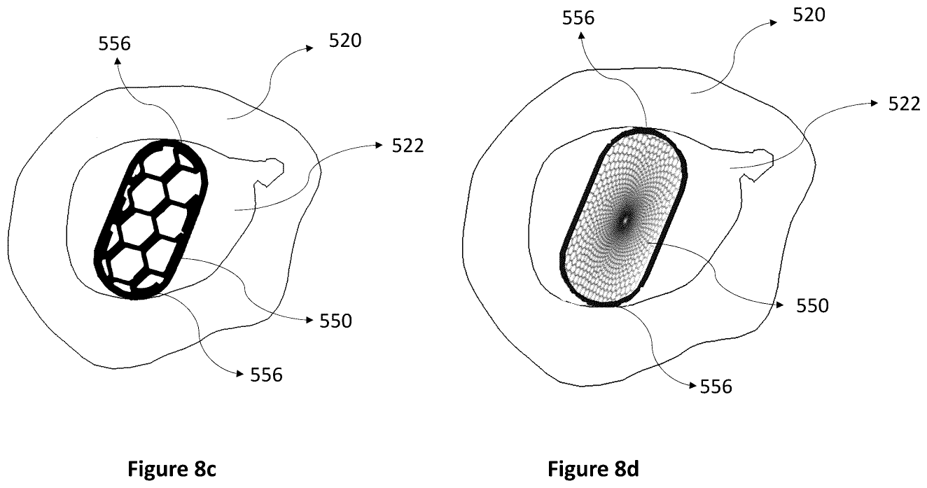

FIGS. 8a to 8d show cross-sectional top views of implants 550 positioned within the medullary canal 522 of the femur bone 520 having different internal lattice structures. FIG. 8a shows an exemplified representation of a fully-custom designed implant having a solid core structure whereby at least the mid-segment geometrical dimensions of the implant cross-section are configured to correspond closely to the geometrical dimensions and shape of the medullary canal 522. FIGS. 8b to 8d show semi-custom presentations of an implant having an ellipsoid cross-section, whereby only certain regions 556 of the mid-segment 552 are in contact with corresponding regions of the medullary canal 522. The implant representation of FIGS. 8a to 8d may be provided with an internal core structure selected from solid fill, isotropic lattice structure, or anisotropic lattice structure.

Step 410, of adapting the design characteristics of implant, may optionally include step 410a of analysing and optimising the mechanical behaviour of the implant. The step 410a involves the Computer Aided Engineering (CAE) analysis of the implant's mechanical behaviour to access the implant's performance. The results are automatically evaluated, the optimization's objective function is calculated and fed into an optimization algorithm. This can be a gradient-based algorithm, such as the Local Descent Algorithm, or a Non-Differential Objective Function, such as the Particle Swarm Optimization Algorithm, depending on whether the objective function can be differentiated at a point or not. The algorithm modifies the implant's design parameters and initiates a new design-analysis-optimization cycle. It is worth noting that variable internal lattice structures, of variable sizes and density, some based on mechanical and some on natural lattices are tried out during this process until the optimum mechanical design is found. This process is repeated until the objective function has been minimized, or until repeated changes in the design offer negligible reductions of the objective function. Once this point is reached, the optimum design for this patient has been found.

The aim of the implant design optimization is the development of optimum hip implants that will attain the following objectives:
1) Elimination of the Stress Shielding effect in the implanted femur, after Total Hip Arthroplasty (THA), aiming at obtaining an implant stress distribution profile similar to the one presented in the intact bone.
2) Minimize the stiffness mismatch between the femur and the stem, which can lead to unnatural stresses in the implanted bone, caused by the load transfer generated due to daily activities. This can be achieved by minimizing the difference of the maximum induced displacement between the intact and the implanted femur.
3) Minimize the mass to volume fraction of the femoral stem (femoral stem's density).

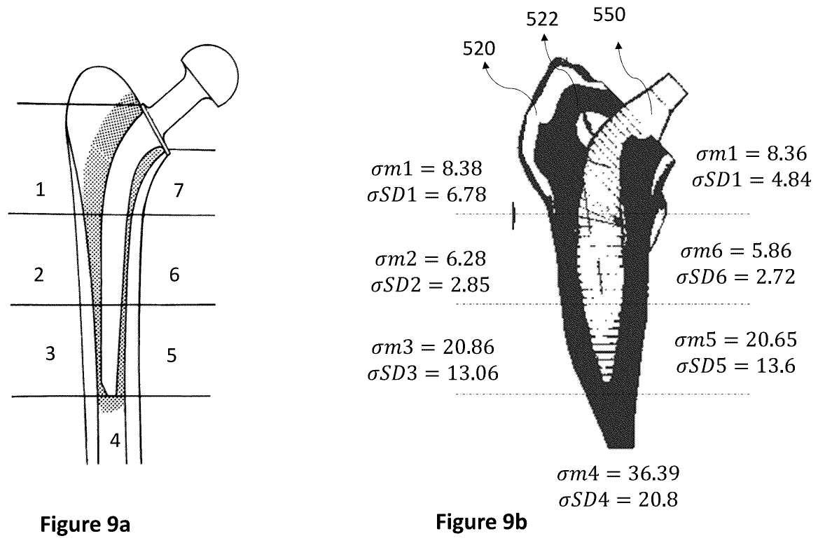

In order to minimize the stress shielding effect, the Von Mises stresses are investigated both in the intact (pre-THA) and the operated femur (after THA). In particular, the objective is to minimize the relative difference between the volume average of the Von Mises stresses (VMS) of the intact and the implanted femur calculated over a region of interest. In particular, the regions of interest are the Gruen zones as shown in FIG. 9a, which are seven zones along which both the intact and the operated femur are separated.

Based on the above, the respective single cost function is formulated as:

$$min f \text{?} = ( \text{?} ) - ( \text{?} ) \qquad Eq . [ 2 ]$$

$$\text{?} \text{indicates text missing or illegible when filed}$$

where σ_{VMS}^{pre-tha} is the volume average of the Von Mises stresses for the elements in a VMS specific Gruen zone of the intact femur and σ_{VMS}^{tha} is the volume average of the VMS stresses for the elements in the respective Gruen zone of the implanted femur, as shown in FIG. 9b.

Concerning the second optimization criterion, in order to eliminate the stiffness mismatch between bone-stem and consequently the stress shielding effect, the aim is to minimize the difference between the maximum displacement exhibited in the intact bone (mainly in the region of the abductor) and the respective one induced in the implanted femur. To reach that goal, the corresponding single cost function is defined as:

$$min f \text{?} = ( \text{?} ) - ( \text{?} ) \qquad Eq . [ 3 ]$$

$$\text{?} \text{indicates text missing or illegible when filed}$$

where Ui^{pre-tha} i is the maximum displacement calculated in a node of the intact bone and Ui^{tha} i is the maximum displacement calculated in a node of the operated femur.

Finally, for achieving minimization of the implant's mass to volume fraction, the single cost function, f m/v, is considered:

$$min f \text{?} = \text{?} \qquad Eq . [ 4 ]$$

$$\text{?} \text{indicates text missing or illegible when filed}$$

Where p^{implant} is the calculated density of a femoral stem design.

Once all the anatomical and implant parameters have been defined, a CAD file is generated at step 412, where the implant dataset is represented using lofts of the cross-sections computed which are joined together forming the 3D shape of the implant. According to the present invention, custom and semi-custom implants designed according to the presented method have the advantage that can only be implanted in a specific desired position within the medullar canal such that the anteversion and/or neck shaft angle of the implant satisfy the patient's femoral bone anatomical characteristics. As such, the present invention prevents misplacement of the implant in the medullary canal during Total Hip Arthroplasty (THA), which may result in incorrect anteversion and/or neck shaft angle that may affect patient conform and implant longevity. Along with the implant, the broach, osteotomy guide, anatomical models of the intact and implanted femur and a 3D PDF guide containing all design information are created, if selected by the surgeon. The information in the generated CAD file may be used by an additive manufacturing system (e.g. a 3D printer) for the manufacturing of the implant and corresponding surgical tools e.g. broaching/rasp and osteotomy guide apparatuses.

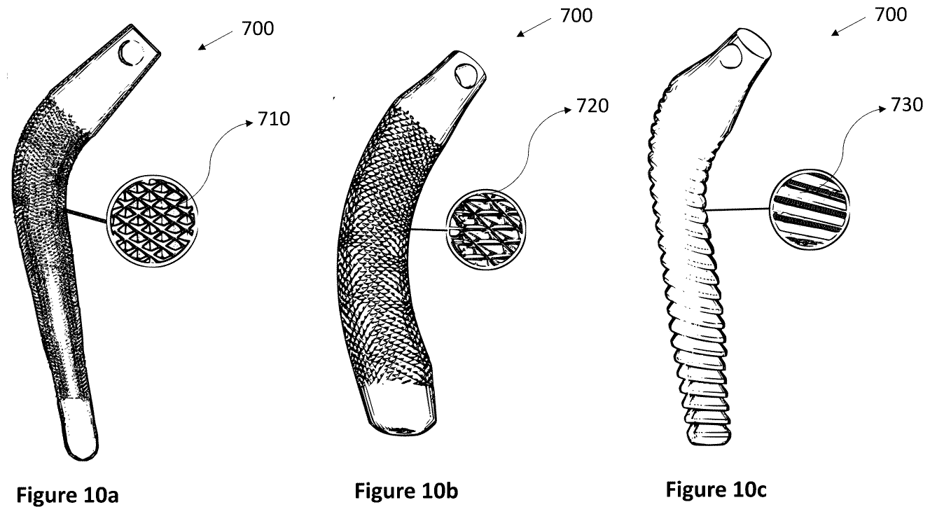

FIGS. 10a to 10c show exemplified representation of different rasp/broaching apparatus as designed according to embodiments of the present invention. The process of cavity preparation by broaching has an impact on the primary stability of uncemented hip stems and on the periprosthetic fracture risk. Insufficient bone-implant contact can result in insufficient primary stability resulting in loosening of the implant, whereas high strains due to an exaggerated press-fit can lead to early bone fracture. If both extremes can be prevented, osseointegration of the prosthesis can take place, leading to safe secondary stability and providing the basis for the longevity of the implant. As such according to embodiments of the present invention, rasps matching as closely as possible to the final implant geometry are generated as shown in FIGS. 10a to 10c, which represent broaches used for sharp extraction, blunt extraction, and compaction. Customised broaches based on the stem's exact geometry are automatically designed in any type and density selected by the surgeon using the digital implant platform 100.

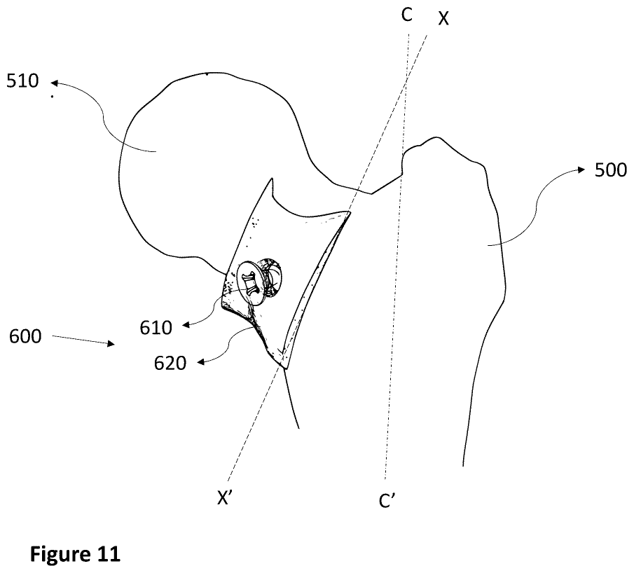

FIG. 11 shows an exemplified implementation of an osteotomy guide apparatus 600 designed according to embodiments of the present invention. The osteotomy guide apparatus 600 is configured to be mounted on the femoral neck isthmus of the femoral bone. The osteotomy apparatus 600 is adapted to match a geometry of the femoral neck so as to provide a guiding plane x-x′ for the osteotomy procedure. The guide 600 comprises a fixation portion 610, which is configured to at least partially surround the femoral neck isthmus of the femoral bone. The design characteristics of the fixation portion 610 are adapted such that the fixation portion is adapted to match the geometrical dimensions of the femoral neck isthmus. The guide 600 may be generated based on information extracted at steps 402 and 404 of the design method of FIG. 2. The fixation portion 610 may be press-fitted on the femur neck isthmus and secured in the desired positioned with one or more securing elements e.g. screw. As such the surgeon is provided with a customised guiding plane for performing the osteotomy that is designed according to the patient's specific femur bone anatomy.

1. A computer implemented method for designing a femoral joint implant, the method comprising the steps of:
generating a three-dimensional graphic representation of a human femoral bone based on a dataset obtained from a computerised tomography, CT, scan of the human pelvis and femoral bone, the graphic representation comprising a representation of a structure of the pelvis, femur and medullary canal of the human bone where the implant is to be positioned;
processing the graphic representation of the human femoral bone to extract a set of cortical bone and medullary canal parameters comprising the shape and geometric dimensions of the medullary canal;
generating a digital model of the implant based on the extracted set of cortical bone and medullary canal parameters, the digital implant model representing an implant comprising a stem having a distal segment, a middle segment, and a proximal segment, wherein the step of generating the digital implant model comprises the step of
adapting design characteristics of one or more segments of the stem of the digital implant model according to the set of cortical bone and medullary canal parameters such that the geometrical parameters of the one or more segments correspond to the geometrical parameters of the medullary canal over a predetermined area of the femoral bone; and
generating a set of digital implant model data representing the digital implant model with the adapted characteristics for use in the fabrication of the femoral implant, and
wherein the step of generating the three-dimensional graphic representation of a patient's bone comprises the steps of:
Processing a DICOM data series comprising the CT scan dataset representing the patient's hip region including the femoral bone;
determining and exclude in the processed DICOM data file artifacts corresponding to Hounsfield below or above a predetermined threshold;
generating, by means of a trained neural network, segmentation masks to represent the femoral bone's cortical parts and the pelvis bone; and
generating a 3D reconstruction of the femoral bone based on the generated segmentation masks and the DICOM series metadata to generate triangulated meshes of the pelvis and femur's surfaces based on a marching cube algorithm.

2. The method of claim 1, wherein the step of adapting the design characteristics of the digital implant model comprises the step of defining one or more regions of the digital implant model stem and adapting the cross-sectional geometrical dimensions and/or shape of the one or more regions to match the cross-sectional shape and geometrical dimensions of corresponding regions of the medullary canal structure, when the femoral joint implant is positioned in the medullary canal.

3. The method of claim 2, wherein the one or more regions are defined between the distal segment and the middle segment of the digital implant model.

4. The method of claim 1, wherein the step of adapting the design characteristics of the digital implant model is based on a set of implant parameters, which are defined and/or selected during the generation of the digital implant model, wherein the values for the set of implant parameters is obtained from the femoral bone dataset and/or provided as input by a user.

5. The method of claim 1, wherein the digital implant model representation is based on ellipsoids and anatomically derived curves connected with lofts, and wherein 3D splines are used to control the shape of the ellipsoids so as to adapt the design characteristics of the digital implant model including the shape and/or the geometrical dimensions of the digital implant model.

6. The method of claim 1, wherein the step of adapting the design characteristics of the digital implant model comprises the step of analysing the mechanical behaviour of the digital implant model at the one or more regions over the predetermined area of the medullary canal based on results obtained from a Finite Element Analysis, FEA, of a femur bone model comprising the designed implant in comparison with corresponding results of an intact femur bone model.

7. The method according to claim 6, wherein the step of adapting the design characteristics of the digital implant model comprises the step of optimising the design characteristics of the digital implant model at the one or more regions by iteratively varying the design characteristics of the digital implant model until the difference between the FEA results obtained for the digital implant model and the intact femur model is minimized, or until repeated changes in the design of the digital implant model offer reductions below a predetermined threshold.

8. The method of claim 7, wherein at each iteration one or more of the design characteristics of the digital implant model is adapted and the resulting mechanical behaviour is analysed to determine if resulting adaptation reduces and/or minimizes an objective function representing the difference between the corresponding FEA results from the digital implant model and the intact femur model.

9. The method of claim 8, wherein the objective function comprises one or more optimisation criteria, the criteria comprising any one or a combination of:
elimination of Stress Shielding effect;
minimization of a stiffness mismatch between the femur and the femoral implant stem;
minimization of a density of the femoral implant stem while maintaining expected loading capacity; and
minimization of a relative micromotion between the implant and the femur.

10. The method of claim 1, wherein the set of design characteristics of the implant model comprise any one or a combination of: geometrical dimensions, shape, internal implant lattice structure, material, and density of the implant.

11. The method of claim 1, wherein the step of generating the digital implant model comprises adapting a surface of a region of the implant middle segment by performing the steps of:
generating a pattern of a trabecular bone cancellous structure surrounding a region of the middle segment implant surface; and
subtracting the pattern from the surface of the implant, to generate a representative pattern of structures corresponding to the cancellous structure of the trabecular bone structure so as to promote osseo-integration, wherein the representative pattern of structures comprises cavities configured to promote growth of trabecular bone structure into the artificial joint implant.

12. The method of claim 1, wherein the set of digital implant model data is a computer aided design, CAD, file at least defining a 3D shape of the implant, wherein the CAD file is outputted to a additive printer for fabrication of the femoral joint implant.

13. A computer system for designing a femoral implant and corresponding broaching and osteotomy guide apparatus, the system comprising:
a user interface running on an electronic device; and
a processing unit configured to perform, based on information and/or instructions received by a user through the user interface, the method according to claim 1.

14. A computer system comprising instructions which when executed by a computer performs the method according to claim 1.
